(19) United States
(12) Patent Application Publication
(10) Pub. No.: US 2022/0330894 A1
(43) Pub. Date: Oct. 20, 2022

(54) CONTACTLESS INTELLIGENT MONITOR AND ITS DETECTION METHOD

(71) Applicant: QUANZHOU NORMAL UNIVERSITY, Quanzhou Fujian (CN)

(72) Inventors: Zhihao CHEN, Quanzhou, Fujian (CN); Cunjiang WU, Quanzhou Fujian (CN); Yanxiang WANG, Quanzhou Fujian (CN); Wenhui WEI,, Quanzhou Fujian (CN); Yunjie RUAN, Quanzhou Fujian (CN); Weitang SUN, Quanzhou Fujian (CN); Huicheng YANG, Quanzhou Fujian (CN)

(73) Assignee: QUANZHOU NORMAL UNIVERSITY, Quanzhou Fujian (CN)

(21) Appl. No.: 17/616,646

(22) PCT Filed: Oct. 14, 2020
(86) PCT No.: PCT/CN2020/120969

§ 371 Date: Dec. 4, 2021

(30) Foreign Application Priority Data
Jul. 29, 2020 (CN) 202010741628.2

(51) Int. Cl. A61B 5/00; A61B 5/0205

(52) U.S. Cl. CPC A61B 5/7203; A61B 5/7225; A61B 5/0205; A61B 5/7221; A61B 2562/223; A61B 5/02416

(57) ABSTRACT

The present invention relates to a contactless intelligent monitor, including: a light source, a coupler, a concave-convex noise reduction unit, a first photodetector, a second photodetector, an MCU and a terminal; the light source, coupler and concave-convex noise reduction unit are connected in sequence; the output of the concave-convex noise reduction unit is connected to the MCU through the first photodetector and the second photodetector respectively; the MCU is connected to the terminal through a communication module. The present invention can effectively reduce the interference and false alarm caused by noises in the measurement process.

### FIELD OF TECHNOLOGY

The present invention relates to the field of optical fiber sensing technology, and in particular to a contactless intelligent monitor and its detection method.

### BACKGROUND

With the progress of optical fiber sensing technology optical fiber sensors have been used to detect human vital sign parameters (such as respiratory rate, heart rate and body movement) without direct contact with body skin. An early US patent, U.S. Pat. No. 6,498,652B1, proposed the use of optical fiber interferometer to detect human vital sign parameters. However, optical fiber interferometer requires a coherent light source and a shielded optical fiber reference arm, which increases the cost and its signal demodulation is relatively complex. The practical and commercial application of such sensors has great challenges. There are still many researches in this field, for example, the recently published paper Noninvasive Monitoring of Vital Signs Based on Highly Sensitive Fiber Optic Mattress. IEEE Sensors J., 20(11): 6182-6190, 2020. Fiber Bragg grating sensor for monitoring vital sign parameters is considered to be a potential sensor and have been extensively studied. Such fiber Bragg grating sensor uses wavelength detection method, the technology is complex, the equipment is expensive, and its practicability and commercialization are also facing great challenges.

In the prior art, a micro bending optical fiber sensor can also be used to detect patient's breathing, heart rate and body movement. This sensor system is simple, low cost, easy to manufacture while having high sensitivity and good robustness, and has been practical and commercialized, and used at home. This sensor is one of the most attractive sensors available, comparable in cost and reliability to conventional electrical sensors and offering advantages not found in electrical sensors, such as anti-electromagnetic interference, rich spectrum characteristics, scalable sensing area and sensitivity. Research into micro bending optical fiber sensor for vital signs has long been overlooked. Like all intelligent hardware, noise interference is the number one problem that needs to be solved for micro bending optical fiber sensor and others.

### SUMMARY

In view of this, the purpose of the present invention is to propose a contactless intelligent monitor and its detection method, which can effectively reduce the interference and false alarm caused by noises in the measurement process.

In order to achieve the above purpose, the present invention is realized with the following solutions: a contactless intelligent monitor, including: a light source, a coupler, a concave-convex noise reduction unit, a first photodetector, a second photodetector, a Microcontroller Unit (MCU) and a terminal; the light source, coupler and concave-convex noise reduction unit are connected in sequence; the output of the concave-convex noise reduction unit is connected to the MCU through the first photodetector and the second photodetector respectively; the MCU is connected to the terminal through a communication module.

Further, the concave-convex noise reduction unit includes a concave-convex noise reduction component, a first transmission optical fiber and a second transmission optical fiber; the first transmission optical fiber and second transmission optical fiber are provided on the upper surface and the lower surface of the concave-convex noise reduction component respectively.

Further, the concave-convex noise reduction unit includes a first concave-convex noise reduction component, a second concave-convex noise reduction component, a first transmission optical fiber and a second transmission optical fiber provided in order from top to bottom; the first transmission optical fiber is provided on the lower surfice of the first concave-convex noise reduction component; the second transmission optical fiber is provided on the lower surface of the second concave-convex noise reduction component.

Further, the concave-convex noise reductionunit comprises a first concave-convex noise reduction component, a second concave-convex noise reduction component, a third concave-convex noise reduction component, a first transmission optical fiber and a second transmission optical fiber provided in order from top to bottom; the first transmission optical fiber is provided between the lower surface of the first concave-convex noise reduction component and the upper surface of the second concave-convex noise reduction component; the second transmission optical fiber is provided between the lower surface of the second concave-convex noise reduction component and the upper surface of the third concave-convex noise reduction component.

Further, the light source uses a light emitting diode or a laser light source.

Further, the transmission optical fiber is a multimode optical fiber or a single-mode optical fiber or a mixture of single-mode optical fiber and multimode optical fiber.

Further, the concave-convex noise reduction component is a resilient sheet body with a concave-convex shape.

A detection method of a contactless intelligent monitor, which comprises the following steps:

Step S1: the light source is divided into incident light 1 and incident light 2 after passing through a coupler and transmitted to a first transmission optical fiber and a second transmission optical fiber respectively;

Step S2: after passing through the first transmission optical fiber and the second transmission optical fiber, the incident light is transmitted to a first photodetector and a second photodetector respectively through a output optical fiber;

Step S3: a photocurrent 1 and a photocurrent 2 are obtained through photodetector conversion and input into MCU;

Step S4: the MCU amplifies, filters, analog-to-digital converts, calculates and analyses the input photocurrent signal;

Step S5: the processed data is transmitted to the terminal through a communication module.

Further, the calculation and analysis are as follows:

Step S41: calculating the respiratory rate RR1 from the signal data of the photocurrent 1 and the respiratory rate RR2 from the signal data of the photocurrent 2; if the error between RR1 and RR2 is less than 3 bpm, the respiratory rate RR=(RR1+RR2)/2; otherwise it is the result of a false alarm and the data is not uploaded to the terminal;

Step S42: calculating the heartbeat frequency from the signal data of the photocurrent 1:

$$f 11 = f + df 11 \\ f 12 = 2 f + df 12 \\ f 13 = 3 f + df 13 \\ … \\ f 1 N = Nf + df 1 N$$

calculating the heartbeat frequency from the signal data of the photocurrent 2:

$$f 21 = f + df 21 \\ f 22 = 2 f + df 22 \\ f 23 = 3 f + df 23 \\ … \\ f 2 N = Nf + df 2 N$$

the heart rate on the path of photocurrent 1 is:

$$HR 11 = f 11 * 60 \\ HR 12 = f 12 * 60 / 2 \\ HR 13 = f 13 * 60 / 3 \\ … \\ HR 1 N = f 1 N * 60 / N$$

the heart rate on the path of photocurrent 2 is:

$$HR 21 = f 21 * 60 \\ HR 22 = f 22 * 60 / 2 \\ HR 23 = f 23 * 60 / 3 \\ … \\ HR 2 N = f 2 N * 60 / N$$

Step S43: calculating the heart rate HR1=(HR11+HR12+HR13+ . . . +HR1N)/N from the signal data of photocurrent 1 and the heart rate HR2=(HR21+HR22+HR23+ . . . +HR2N)/N from the signal data of photocurrent 2; if the error between HR1 and HR2 is less than the preset value, the heart rate HR=(HR1+HR2)/2; otherwise it is the result of a false alarm and the data is not output to the terminal.

Compared with the prior art, the present invention has the following beneficial effects:

1. The invention can effectively reduce the interference and false alarm caused by noises in the measurement process.

2. The invention has lowcost and is easy to apply and popularize.

### BRIEF DESCRIPTION OF THE DRAWINGS

FIG. 1 is a schematic diagram of the system structure of embodiment 1 of the present invention;

FIG. 2 is a schematic diagram of the system structure of embodiment 2 of the present invention;

FIG. 3 is a schematic diagram of the system structure of embodiment 3 of the present invention;

FIG. 4 is a schematic diagram of the structure of the concave-convex noise reduction unit of embodiment 1 of the present invention;

FIG. 5 is a schematic diagram of the structure of the concave-convex noise reduction unit of embodiment 2 of the present invention;

FIG. 6 is a schematic diagram of the structure of the concave convex noise reduction unit of embodiment 3 of the present invention.

### DESCRIPTION OF THE EMBODIMENTS

The present invention will be further described below in conjunction with the accompany drawings and embodiments.

### EXAMPLE 1

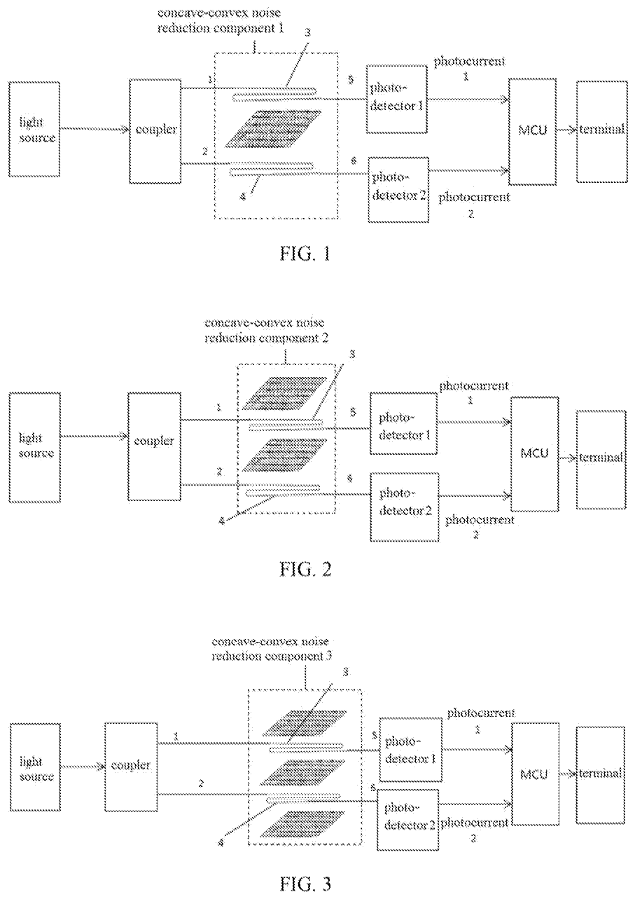

Referring to FIG. 1, this embodiment provides a contactless intelligent monitor, including: a light source, incident optical fiber 1 and incident optical fiber 2, transmission optical fiber 3 and transmission optical fiber 4, outgoing optical fiber 5 and outgoing optical fiber 6, concave-convex noise reduction component 1, photodetector 1 and photodetector 2, an MCU, and a terminal.

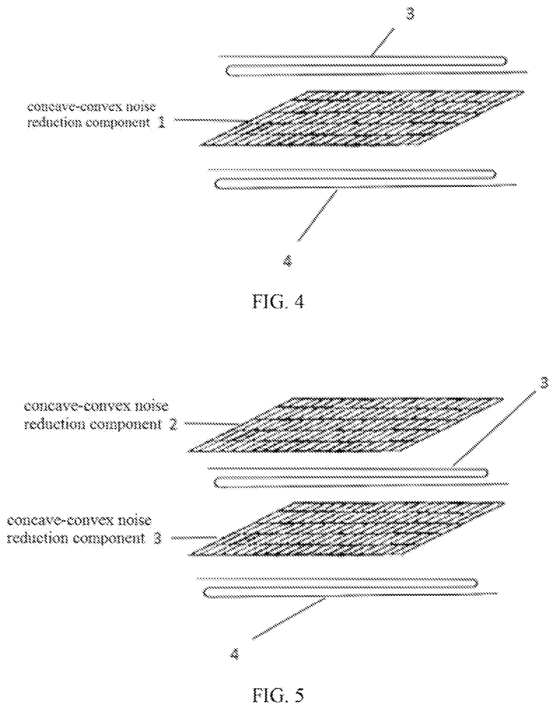

Preferably, referring to FIG. 4, in this embodiment, the above-mentioned transmission optical fibers 3 and 4 are placed on the upper and lower surfaces of the. concave-convex noise reduction component 1 respectively.

In this embodiment, the light source enters the incident optical fibers 1 and 2 respectively after passing through the 1×2 coupler, while the incident optical fibers 1 and 2 are transmitted through the transmission optical fibers 3 and 4 respectively. Then they are input to the photodetector 1 and photodetector 2 through the output optical fibers 5 and 6. After passing through the photodetector 1 and photodetector 2, the light waves in the optical fibers 5 and 6 are converted into photocurrent 1 and photocurrent 2. In the MCU, the photocurrent goes through a series of processing such as amplification, filtering, analog-to-digital conversion, and calculation.

In this embodiment, preferably, the calculation processing is specified as follows:

Calculating the respiratory rate RR1 from the photocurrent 1 and the respiratory rate RR2 from the photocurrent 2. If the error between RR1 and RR2 is less than 3 bpm, the respiratory rate RR=(RR1+RR2)/2. Otherwise it is the result of a false alarm. Ensure that the accuracy of the measured respiratory rate is within a certain range and no false alarm occurs.

Calculating the heartbeat frequency from the signal data of the photocurrent 1:

$$f 11 = f + df 11 \\ f 12 = 2 f + df 12 \\ f 13 = 3 f + df 13 \\ … \\ f 1 N = Nf + df 1 N$$

Calculating the heartbeat frequency from the signal data of the photocurrent 2:

$$f 21 = f + df 21 \\ f 22 = 2 f + df 22 \\ f 23 = 3 f + df 23 \\ … \\ f 2 N = Nf + df 2 N$$

The heart rate on the path of photocurrent 1 is:

$$HR 11 = f 11 * 60 \\ HR 12 = f 12 * 60 / 2 \\ HR 13 = f 13 * 60 / 3 \\ … \\ HR 1 N = f 1 N * 60 / N$$

The heart rate on the path of photocurrent 2 is:

$$HR 21 = f 21 * 60 \\ HR 22 = f 22 * 60 / 2 \\ HR 23 = f 23 * 60 / 3 \\ … \\ HR 2 N = f 2 N * 60 / N$$

Calculating the heart rate HR1=(HR11+HR12+HR13+ . . . +HR1N)/N from the path of photocurrent 1 and the heart rate HR2=(HR21+HR22+HR23+ . . . +HR2N)/N from the path of photocurrent 2. If the error between HR1 and HR2 is less than the preset value, such as 3 bpm, the heart rate HR=(HR1+HR2)/2. Otherwise it is the result of a false alarm and the data is not output to the terminal. Where N is a positive integer, at least 1, and usually not more than 10.

Preferably, in this embodiment the calculation results are transmitted to the terminal host computer via wired or wireless devices such as Bluetooth, and the host computer software performs various processing, analysis, display and alarming of the received results in terms of applications.

### EXAMPLE 2

Referring to FIG. 2, this embodiment provides a contactless intelligent monitor, including: a light source, incident optical fiber 1 and incident optical fiber 2, concave-convex noise reduction component 2, concave-convex noise reduction component 3, outgoing optical fiber 5 and outgoing optical fiber 6, photodetector 1 and photodetector 2, an MCU, and a terminal.

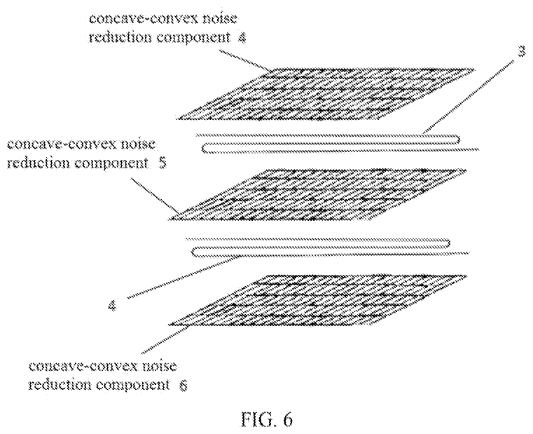

Referring to FIG. 5, in this embodiment, preferably, the transmission optical fiber 3 is placed between the lower surface of the concave-convex noise reduction component 2 and the upper surface of the concave-convex noise reduction component 3, the transmission optical fiber 4 is placed on the lower surface of the concave-convex noise reduction component 3.

The light source enters the incident optical fibers 1 and 2 respectively after passing through the 1×2 coupler, while the incident optical fibers 1 and 2 are transmitted through the transmission optical fibers 3 and 4 respectively. Then they are input to the photodetector 1 and photodetector 2 through the output optical fibers 5 and 6. After passing through the photodetector 1 and photodetector 2, the light waves in the optical fibers 5 and 6 are converted into photocurrent 1 and photocurrent 2. In the MCU, the photocurrent goes through a series of processing such as amplification, filtering, analog-to-digital conversion, and calculation.

### EXAMPLE 3

Referring to FIG. 3, this embodiment provides a contactless intelligent monitor, including: a light source, incident optical fiber 1 and incident optical fiber 2, concave-convex noise reduction component 4, concave-convex noise reduction component 5, concave-convex noise reduction component 6, outgoing optical fiber 5 and outgoing optical fiber 6, photodetector 1 and photodetector 2, an MCU, and a terminal.

Referring to FIG. 6, in this embodiment, preferably, the transmission optical fiber 3 is placed between the lower surface of the concave-convex noise reduction component 4 and the upper surface of the concave-convex noise reduction component 5; the transmission optical fiber 4 is placed between the lower surface of the concave-convex noise reduction component 5 and the upper surface of the concave-convex noise reduction component 6;

The light source enters the incident optical fibers 1 and 2 respectively after passing through the 1×2 coupler, while the incident optical fibers 1 and 2 are transmitted through the transmission optical fibers 3 and 4 respectively. Then they are input to the photodetector 1 and photodetectot 2 through the output optical fibers 5 and 6. After passing through the photodetector 1 and photodetector 2, the light waves in the optical fibers 5 and 6 are convened into photocurrent 1 and photocurrent 2. In the MCU, the photocurrent goes through a series of processing such as amplification, filtering, analog-to-digital conversion, and calculation.

In this embodiment, the preferred concave-convex noise reduction component may be made of a material with a concave-convex structure such as mesh screen, filter, gauze, cloth, plastic mesh, etc.

A calculation example is given below:

Table 1 shows the results of respiratory and heart frequency calculations for a volunteer. The respiratory frequency calculated from photocurrent 1 is 0.244 hz, and the respiratory frequency calculated from photocurrent 2 is 0.245 hz. The respiratory rate RR1=0.244*60=14.64 bpm and RR2=0.245*60=14.7 bpm. The error of RR1 and RR2 is less than 3 bpm, then the respiratory rate RR=(RR1+RR2)/2=14.67 bpm, as shown in Table 2.

According to table 2, the fundamental and harmonic frequencies of heartbeat calculated from photocurrent 1 are 1.12 hz, 2.29 hz and 3.42 hz; the fundamental frequencies of heartbeat calculated from photocurrent 2 are 1.12 hz, 2.25 hz and 3.4 hz. So the heart rate

HR1=(1.12+2.29/2+3.42/3)*60/3=68.1 bpm

The heart rate

HR2=(1.12+2.25/2+3.42/3)*60/3=67.7 bpm

The errors of both HR1 and HR2 are within 1 bpm (less than 3 bpm), according to the method of this embodirmnt, no false alarm will occur.

TABLE 1
Respiratory and heart frequency calculations of a volunteer

| | | | Heartbeat | Heartbeat |
|---|---|---|---|---|
| | | Heartbeat | second | third |
| | Respiratory | fundamental | harmonic | harmonic |
| | frequency | frequency | frequency | frequency |
| | (Hz) | (Hz) | (Hz) | (Hz) |
| Photocurrent 1 | 0.244 | 1.12 | 2.29 | 3.42 |
| Photocurrent 2 | 0.245 | 1.12 | 2.25 | 3.42 |

TABLE 2
Respiratory rate and heart rate calculations of a volunteer

| | | | Heart rate | Heart rate |
|---|---|---|---|---|
| | | Heart rate | corresponding | corresponding |
| | | corresponding | to heartbeat | to heartbeat |
| | | to heartbeat | second | third |
| | Respiratory | fundamental | harmonic | harmonic |
| | rate (bpm) | wave (bpm) | wave (bpm) | wave (bpm) |
| Photocurrent 1 | 14.64 | 67.2 | 68.7 | 68.4 |
| Photocurrent 2 | 14.7 | 67.2 | 67.5 | 68.4 |

Note:
Respiratory rate (bpm) = respiratory frequency × 60
Heart rate (bpm) corresponding to heartbeat fundamental wave = heartbeat fundamental frequency × 60
Heart rate (bpm) corresponding to heartbeat second harmonic wave = (heartbeat second harmonic frequency/2) × 60
Heart rate (bpm) corresponding to heartbeat third harmonic wave = (heartbeat third harmonic frequency/3) × 60

In this embodiment, there is at least 1 concave-convex noise reduction component, and the more the concave-convex noise reduction components, the better the noise reduction effect. At this time, it is necessary to use multiplex transmission optical fibers, 1× multiplex fiber coupler, multiple photodetectors.

The above-mentioned is only a better implementation of the invention, all the equal changes and modifications made according to the scope of the patent application of the invention shall be covered bv the present invention.

What is claimed is:

1. A contactless intelligent monitor, characterized in that it includes: a light source, a coupler, a concave convex noise reduction unit, a first photodetector, a second photodetector, an MCU and a terminal; the light source, coupler and concave-convex noise reduction unit are connected in sequence; the output of the concave-convex noise reduction unit is connected to the MCU through the first photodetector and the second photodetector respectively; the MCU is connected to the terminal through a communication module.

2. A contactless intelligent monitor according to claim 1, characterized in that: the concave-convex noise reduction unit includes a concave-convex noise reduction component, a first transmission optical fiber and a second transmission optical fiber; the first transmission optical fiber and second transmission optical fiber are provided on the upper surface and the lower surface of the concave-convex noise reduction component respectively.

3. A contactless intelligent monitor according to claim 1, characterized in that: the concave-convex noise reduction unit includes a first concave-convex noise reduction component, as second concave-convex noise reduction component a first transmission optical fiber and a second transmission optical fiber provided in order from top to bottom; the first transmission optical fiber is provided on the lower surface of the first concave-convex noise reduction component; the second transmission optical fiber is provided on the lower surface of the second concave-convex noise reduction component.

4. A contactless intelligent monitor according to claim 1, characterized in that: the concave-convex noise reduction unit comprises a first concave-convex noise reduction component, a second concave-convex noise reduction component, a third concave-convex noise reduction component, a first transmission optical fiber and a second transmission optical fiber provided in order from top to bottom; the first transmission optical fiber is provided between the lower surface of the first concave-convex noise reduction component and the upper surface of the second concave-convex noise reduction component; the second transmission optical fiber is provided between the lower surface of the second concave-convex noise reduction component and the upper surface of the third concave-convex noise reduction component.

5. A contactless intelligent monitor according to claim 1, characterized in that: the light source uses a light emitting diode or a laser light source.

6. A contactless intelligent monitor according to any one of claims 2-4, characterized in that: the transmission optical fiber is a multimode optical fiber or a single-mode optical fiber or a mixture of single-mode optical fiber and multimode optical fiber.

7. A contactless intelligent monitor according to any one of claims 2-4, characterized in that: the concave-convex noise reduction component is a resilient sheet body with a concave-convex shape.

8. A detection method of a contactless intelligent monitor, characterized in that it comprises the following steps:
Step S1: the light source is divided into incident light 1 and incident light 2 after passing through a coupler and transmitted to a first transmission optical fiber and a second transmission optical fiber respectively;
Step S2: after passing through the first transmission optical fiber and the second transmission optical fiber, the incident light is transmitted to a first photodetector and a second photodetector respectively through a output optical fiber;
Step S3: a photocurrent 1 and a photocurrent 2 are obtained through photodetector conversion and input into MCU;
Step S4: the MCU amplifies, filters, analog-to-digital converts, calculates and analyses the input photocurrent signal;
Step S5: the processed data is transmitted to the terminal through a communication module.

9. A detection method of a contactiess intelligent monitor according to claim 8, characterized in that the calculation and analysis are as follows:
Step S41: calculating the respiratory rate RR1 from the signal data of the photocurrent 1 and the respiratory rate RR2 from the signal data of the photocurrent 2; if the error between RR1 and RR2 is less than 3 bpm, the respiratory rate RR=(RR1+RR2)/2; otherwise it is the result of a false alarm and the data is not uploaded to the terminal;
Step S42: calculating the heartbeat frequency from the signal data of the photocurrent 1:
$${f}_{11} = f + {df}_{11} \\ {f}_{12} = 2 f + {df}_{12} \\ {f}_{13} = 3 f + {df}_{13} \\ … \\ {f}_{1 N} = Nf + {df}_{1 N}$$
calculating, the heartbeat frequency from the signal data of the photocurrent 2:
$${f}_{21} = f + {df}_{21} \\ {f}_{22} = 2 f + {df}_{22} \\ {f}_{23} = 3 f + {df}_{23} \\ … \\ {f}_{2 N} = Nf + {df}_{2 N}$$
the heart rate on the path of photocurrent 1 is:
$${HR}_{11} = {f}_{11} * 60 \\ {HR}_{12} = {f}_{12} * 60 / 2 \\ {HR}_{13} = {f}_{13} * 60 / 3 \\ … \\ {HR}_{1 N} = {f}_{1 N} * 60 / N$$
the heart rate on the path of photocurrent 2 is:
$${HR}_{21} = {f}_{21} * 60 \\ {HR}_{22} = {f}_{22} * 60 / 2 \\ {HR}_{23} = {f}_{23} * 60 / 3 \\ … \\ {HR}_{2 N} = {f}_{2 N} * 60 / N$$
Step S43: calculating the heart rate HR1=(HR₁₁+HR₁₂+HR₁₃+ . . . +HR_{1N})/N from the signal data of photocurrent 1 and the heart rate HR2=(HR₂₁+HR₂₂+HR₂₃+ . . . +HR_{2N})/N from the signal data of photocurrent 2; if the error between HR1 and HR2 is less than the preset value, the heart rate HR=(HR1+HR2)/2; otherwise it is the result of a false alarm and the data is not output to the terminal.
